(19) United States
(12) Patent Application Publication
(10) Pub. No.: US 2025/0163131 A1
(43) Pub. Date: May 22, 2025

(54) SFASL FOR INHIBITING RPE CELL DEATH AND ASSOCIATED DISORDERS

(71) Applicant: ONL Therapeutics, Inc., Ann Arbor, MI (US)

(72) Inventors: Andrew KOCAB, Farmington Hills, MI (US); David ZACKS, Ann Arbor, MI (US)

(21) Appl. No.: 18/724,563

(22) PCT Filed: Oct. 26, 2022
(86) PCT No.: PCT/US2022/078735

§ 371 Date: Jun. 26, 2024

Related U.S. Application Data
(60) Provisional application No. 63/294,044, filed on Dec. 27, 2021.

(51) Int. Cl. C07K 14/705; A61K 9/00; A61K 38/00; A61K 48/00; A61P 27/02; C12N 15/86

(52) U.S. Cl. CPC C07K 14/70575; A61K 9/0048; A61K 48/0058; A61P 27/02; C12N 15/86; A61K 38/00; C12N 2750/14143

(57) ABSTRACT

Provided and described herein are methods of treating retinal pigment epithelial (RPE) cell death in an eye of an individual. In certain instances, the method comprises administering a sFasL therapeutic to the eye, wherein the sFasL therapeutic increases an amount of sFasL polypeptide in the eye. Also provided is the use of a nucleic acid molecule comprising a nucleic acid sequence encoding a soluble Fas ligand (sFasL) polypeptide for the treatment of retinal pigment epithelial (RPE) cell death.

### CROSS-REFERENCE

This application claims priority to U.S. Provisional Patent Application No. 63/294,044, filed on Dec. 27, 2021, which is herein incorporated by reference in its entirety for all purposes.

### STATEMENT OF GOVERNMENT RIGHTS

This invention was made with government support under R42EY029625 awarded by the National Institutes of Health. The government has certain rights in the invention.

### SEQUENCE LISTING

The instant application contains a Sequence Listing which has been submitted electronically in XML format and is hereby incorporated by reference in its entirety. The XML copy, created on Oct. 25, 2022, is named 58109_709_601_SL.xml and is 6,101 bytes in size.

### BACKGROUND

Ocular diseases and disorders associated with and/or characterized by retinal epithelial (RPE) cell loss (e.g., macular degeneration) are among the leading causes of irreversible blindness in the developed world. The risk of developing retinal epithelial cell loss-related diseases such as macular degeneration increases with age, and as the global population continues to grow older, the number of affected persons is predicted to steadily increase. The exact pathogenesis of RPE cell death and associated conditions such as macular degeneration is unclear, but a number of risk factors have been identified. Such factors include environmental factors such as cigarette smoking and high fat diets, and/or genetic factors such as gender, race, and polymorphisms. Under normal homeostatic physiology the eye is able to tolerate these stressors; however, if enough stressors accumulate over time or with enough intensity, the compensatory protective pathways become overwhelmed, resulting in dysfunction of the retinal pigment epithelium (RPE) and buildup of cellular debris. Debris accumulation under the RPE and retina and is clinically apparent as drusen or pseudodrusen, respectively. In end-stage disease, RPE cells undergo programmed cell death forming patches of RPE degeneration known as geographic atrophy (GA). RPE cell death subsequently leads to the loss of trophic support for the photoreceptor cells and results in photoreceptor cell death. Loss of the RPE and photoreceptors ultimately leads to irreversible central vision loss.

### SUMMARY

There is a large unmet medical need for a therapy to prevent and treat diseases and disorders associated retinal pigment epithelial (RPE) cell death and the growth of atrophic and/or necrotic patches within the eye (i.e., a hallmark of advanced dry AMD). Therapeutic approaches have generally targeted various pathways, including the complement system, mitochondrial stress, the visual cycle, among others. While some of these approaches have progressed to clinical development, they have exhibited limitations such as limited impact on the progression of the disease and the need for frequent injection. These limitations suggest that alternative therapeutic targets are needed.

Provided and described herein are compositions and methods useful for treating RPE cell death, photoreceptor cell death associated with or resulting from RPE cell death, and/or diseases associated with or resulting from RPE cell death comprises increasing an amount of a sFasL polypeptide within the eye of an individual (e.g., within retinal tissue). The provided and described compositions and methods are based on the discovery that Fas inactivation and/or inhibition prevents and/or reduces RPE cell loss and protects RPE architecture within the eye of macular degeneration models (a disorder associated with RPE cell death). Furthermore, the provided and described compositions and methods are based on the discovery that Fas inactivation in a physiological state characterized by RPE cell death can be achieved by increasing an amount of soluble Fas ligand (sFasL), thereby in preventing and/or inhibiting RPE cell loss and protecting RPE architecture within the eye of macular degeneration models (a disorder associated with RPE cell death). The described compositions are useful for treating RPE cell death and diseases and disorders associated therewith.

Provided and described herein are methods of treating retinal pigment epithelial (RPE) cell death in an eye of an individual, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for the treatment of retinal pigment epithelial (RPE) cell death.

In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof.

In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration (AMD). In certain embodiments, the age-related macular degeneration comprises wet macular degeneration (e.g., neovascular macular degeneration) or dry macular degeneration. In some embodiments, the macular degeneration comprises or is characterized by geographic atrophy. In some embodiments, the macular degeneration comprises dry macular degeneration. In some embodiments, the disorder is retinitis pigmentosa. In some embodiments, the disorder is Stargardt's disease.

Further provided and described herein are methods of treating photoreceptor cell death in an eye of an individual having retinal pigment epithelial (RPE) cell death, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for the treatment of retinal pigment epithelial (RPE) cell death for treating photoreceptor cell death in an eye of an individual having retinal pigment epithelial (RPE) cell death. In some embodiments, photoreceptor cell death comprises a decrease in electrical activity of photoreceptor cells (e.g., as measured by electroretinogram), a decrease in photoreceptor density (e.g., as measured by adaptive optics scanning laser ophthalmoscopy), or a combination thereof.

In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof.

In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy. In some embodiments, the disorder is retinitis pigmentosa. In some embodiments, the disorder is Stargardt's disease.

Provided and described herein are methods of treating macular degeneration in an eye of an individual, the method comprising: administering to the eye a nucleic acid molecule comprising a nucleic acid sequence encoding a soluble Fas ligand (sFasL) polypeptide. Also provided herein is the use of a nucleic acid molecule comprising a nucleic acid sequence encoding a soluble Fas ligand (sFasL) polypeptide for the treatment of macular degeneration.

In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy.

Provided and described herein are methods of treating retinitis pigmentosa in an eye of an individual, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for treating retinitis pigmentosa in an eye of an individual.

Provided and described herein are methods of treating Stargardt's disease in an eye of an individual, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for treating Stargardt's disease in an eye of an individual.

In some embodiments, the sFasL polypeptide comprises a Fas ligand (FasL) polypeptide comprising (i) an amino acid sequence having at least 90% sequence identity to SEQ ID NO: 1 and (ii) a truncation at an N-terminus of the FasL polypeptide. In some embodiments, the sFasL polypeptide comprises (i) an amino acid sequence having at least 90% sequence identity to SEQ ID NO: 1 and (ii) a truncation at an N-terminus of the FasL polypeptide. In some embodiments, the truncation comprises a deletion of amino acids 1 to about 130, amino acids 1 to about 125, amino acids 1 to about 120, or amino acids 1 to about 115 from SEQ ID NO: 1. In some embodiments, the sFasL polypeptide comprises an amino acid sequence at least 90% sequence identity to SEQ ID NO: 2. In some embodiments, the sFasL polypeptide comprises SEQ ID NO: 2. In some embodiments, the sFasL polypeptide consists of an amino acid sequence as set forth in SEQ ID NO: 2.

In some embodiments, the nucleic acid sequence comprises a sequence identity of at least 90% to SEQ ID NO: 3 and encodes the sFasL polypeptide comprising (i) SEQ ID NO: 2 or (ii) an amino acid sequence at least 90% sequence identity to SEQ ID NO: 2. In some embodiments, the nucleic acid sequence comprises SEQ ID NO: 3. In some embodiments, the nucleic acid sequence is configured for expression in one or more cells in the eye of the individual. In some embodiments, the nucleic acid molecule comprises a promoter comprising a promoter sequence, and wherein the promoter sequence is operably linked to the nucleic acid sequence encoding the soluble sFasL polypeptide. In some embodiments, the promoter is a ubiquitous promoter. In some embodiments, the promoter is a tissue-specific promoter or a cell specific promoter. In some embodiments, the promoter promotes expression of a transgene in one or more cells within the retina (e.g., retinal pigment epithelial cells, retinal ganglion cells, photoreceptors, immune cells, etc.). In some embodiments, the soluble sFasL polypeptide comprises a secretion sequence.

In some embodiments, the nucleic acid molecule is a nucleic acid expression vector. In some embodiments, the nucleic acid expression vector is a viral vector. In some embodiments, the viral vector is selected from the group consisting of: an adenovirus vector, an adeno-associated virus vector, a lentivirus vector, a herpesvirus vector, a poxvirus vector, a baculovirus vector, a papillomavirus vector, and a papovavirus vector. In some embodiments, the viral vector comprises an adenovirus vector. In some embodiments, the viral vector comprises an adeno-associated virus vector. In some embodiments, the viral vector comprises a lentivirus vector. In some embodiments, the viral vector comprises a herpesvirus vector. In some embodiments, the viral vector comprises a poxvirus vector. In some embodiments, the viral vector comprises a baculovirus vector. In some embodiments, the viral vector comprises a papillomavirus vector. In some embodiments, the viral vector comprises a papovavirus vector. In some embodiment the viral vector comprises an AAV2, AAV5, AAV8, AAV9 viral vector or a chimera thereof.

In some embodiments, a viral particle or viral-like particle comprising the viral vector is administered to the eye. In some embodiments, the viral-like particle is an AAV capsid protein. In some embodiments, the AAV capsid protein comprises an AAV2 capsid sequence, an AAV5 capsid sequence, an AAV8 capsid sequence, and or an AAV9 capsid sequence. In some embodiments, the a non-viral vector (e.g., a liposome) comprising the expression vector is administered to the eye.

In some embodiments, the nucleic acid molecule (e.g., the viral vector) is administered to the vitreous of the eye. In some embodiments, the AAV is administered via intravitreal injection, intracameral injection, subretinal injection, injection into the suprachoroidal space, injection in the subtenons space, subconjunctival injection, retrobulbar injection, peribulbar injection, microneedle injection, subretinal injection, or subretinal infusion.

Further provided and descried herein are methods of treating retinal pigment epithelial (RPE) cell death or a disorder associated therewith in an eye of an individual, the method comprising: administering a sFasL therapeutic to the eye, wherein the sFasL therapeutic increases an amount of sFasL polypeptide in the eye. In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof. In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy. In some embodiments, the disorder is retinitis pigmentosa. In some embodiments, the disorder is Stargardt's disease.

Provided and described herein are methods of treating photoreceptor cell death in an eye of an individual having retinal pigment epithelial (RPE) cell death, the method comprising: administering a sFasL therapeutic to the eye, wherein the sFasL therapeutic increases an amount of sFasL polypeptide in the eye. In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof. In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy. In some embodiments, the disorder is retinitis pigmentosa. In some embodiments, the disorder is Stargardt's disease.

In some embodiments, the sFasL polypeptide comprises SEQ ID NO: 3. In some embodiments, the sFasL therapeutic comprises the sFasL polypeptide. In some embodiments, the sFasL therapeutic comprises a nucleic acid molecule encoding the sFasL polypeptide. In some embodiments, the sFasL therapeutic comprises a composition comprising a nucleic acid molecule encoding the sFasL polypeptide and carrier comprising the nucleic acid molecule encoding the sFasL polypeptide.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows data demonstrating that Fas inactivation inhibits RPE cell death and preserves RPE architecture in a macular degeneration model.

FIG. 2 shows data demonstrating that Fas inactivation reduces immune cell activation in the retina.

FIG. 3 shows data demonstrating that an AAV vector configured to express sFasL in the retina increases sFasL in the retina.

FIG. 4 shows data demonstrating that an AAV vector configured to express sFasL in the retina inhibits RPE cell death and preserves RPE architecture in a macular degeneration model.

FIG. 5 shows data demonstrating that an AAV vector configured to express sFasL in the retina reduces immune cell activation in the retina.

### DETAILED DESCRIPTION

Cell death is generally classified into either apoptosis or necrosis. Apoptosis is a controlled form of cell death, and “non-inflammatory” as it does not activate a local inflammatory response characterized by macrophage infiltration. In contrast, necrosis has typically thought to be an uncontrolled process that usually involved a large field of dying cells that induces a more robust inflammatory response. Although apoptosis and necrosis are morphologically distinct, further studies have shown that these processes cannot always be easily distinguished based on histological categorization alone as there are similarities such as: chromatin condensation, mitochondrial permeability, and DNA degradation. Furthermore, aspects of necrosis that were thought to be passive and uncontrolled processes have since been associated with specific signaling pathways, indicating that under certain circumstances, necrosis may be more actively regulated than previously thought. With this updated insight, apoptosis and necrosis are now considered to exist as counterparts on the same continuum, rather than being dichotomous opposites (referred to as necroptosis). Like apoptosis, necroptosis is a form of regulated cell death with key differences. Briefly, apoptosis is often divided into an intrinsic and extrinsic pathway. The intrinsic pathway is activated by insults such as excessive reactive oxygen species, radiation, DNA damage, or oncogene activation. These cellular insults trigger cytochrome c release from the mitochondria, which activates a cascade of caspase cleavage and ultimately the degradation of cellular components. The extrinsic pathway is activated when ligands bind their respective transmembrane receptors in the tumor necrosis factor (TNF) superfamily such as Fas, TNFR1, and TRAILR1. The receptors then oligomerize and recruit cytoplasmic factors that form the death inducing signaling complex (DISC), which in turn activates additional downstream factors. The extrinsic and intrinsic pathways converge at caspase-3/6/7, which along with other “executioner” caspases results in the degradation of intracellular substrates, leading to the death of the cell.

Apoptosis and necroptosis appear to be the main mechanisms of the cell death pathway diseases and disorders associated with retinal pigment epithelial cell death (e.g., as in macular degeneration). For example, photoreceptor and RPE cells in macular degeneration eyes stain positive for DNA fragmentation (i.e., were positive for terminal deoxynucleotidyl transferase dUTP nick end labeling—TUNEL), suggesting the activation of apoptotic cell death pathway and that the Fas/FasL pathway was the upstream regulator of apoptosis in macular degeneration eyes.

In certain instances, pyroptosis may also play a role in RPE atrophy in AMD. Inflammasome activity. Evidence of pyroptosis-like NLRP3 inflammasome activation has been found in eyes of patients with GA or neovascular AMD. RPE cells exposed to oxidative stress undergo cell death through the pyroptosis pathway if cells are grown in culture medium that primed inflammasome activation. Conversely, when capsase-1 activity is inhibited in these cells, cell death is decreased, suggesting that pyroptosis is a pathway of RPE cell death in response to oxidative stress. Furthermore, the chronic inflammation characteristic of drusen accumulation in aged human retinas shows evidence of both pyroptosis and apoptosis, suggesting that the two pathways may be acting in parallel in RPE cell death.

Members of the TNFR superfamily such as Fas, can also activate the necroptotic pathway. However, unlike apoptosis, necroptosis is independent of caspase activity and instead depends on receptor interacting protein kinases (RIPKs) to trigger a signaling cascade that results in activation of membrane-damaging proteins and lytic cell death. It is the activation state of caspase-8 that generally shifts the balance from apoptosis to necroptosis, as caspase-8 normally cleaves RIPK1 and RIPK3 and prevents its downstream effects. In contrast to apoptosis, necroptosis is inherently inflammatory due to its lytic nature. Despite the differences in the apoptotic and necroptotic molecular pathways, in practical application there is no single test that unequivocally distinguishes the two. Instead, both morphologic and biochemical data must be integrated to make the discrimination.

As mentioned above, there is a large unmet medical need for a therapy to prevent the formation and growth of the atrophic and/or necrotic patches (i.e., the hallmark of advanced dry AMD). Therapeutic approaches have generally targeted various pathways, including the complement system, mitochondrial stress, the visual cycle, among others. While some of these approaches have progressed to clinical development, they have exhibited limitations such as limited impact on the progression of the disease and the need for frequent injection. These limitations suggest that alternative therapeutic targets are needed. However, several approaches have been undertaken (caspase inhibitors, etc.) but had only limited success due to intracellular shunting of the death signal. As described herein, the compositions and methods provide a solution to how to effectively treat retinal pigment epithelial (RPE) cell death, photoreceptor cell death associated with or resulting from RPE cell death, and diseases and disorders associated with RPE cell death by targeting inhibition of the Fas signaling pathway by sFasL.

Fas Ligand (FasL) generally refers to and encompasses a 40 kDa type II transmembrane protein of the TNF family, originally identified by its capacity to induce apoptosis in Fas receptor positive cells. FasL can be expressed as a membrane-bound protein (mFasL), or cleaved and released as a soluble protein (sFasL). The human FasL gene encodes for a 281 amino acid protein. In some embodiments, the human FasL gene encodes a protein comprising the amino acid sequence of SEQ ID NO: 1. In some embodiments, FasL GenBank Accession Number NM_000639.2, NCBI Gene ID 356, and/or Uniprot Number P48023.

Soluble Fas Ligand (sFasL) generally refers to and includes the human sFasL protein corresponding to amino acid residues 127-281 of human FasL. In some embodiments, sFasL comprises the amino acid sequence of SEQ ID NO: 2. In some embodiments, the human sFasL protein is encoded by nucleic acids set forth in SEQ ID NO: 3.

Provided and used in the methods described herein are nucleic acid molecules comprising a nucleic acid sequence encoding a sFasL polypeptide. In some embodiments, a sFasL polypeptide comprises the amino acid sequence of SEQ ID NO: 2. In some embodiments, a sFasL polypeptide consists of the amino acid sequence of SEQ ID NO: 2. In some embodiments, the sFasL polypeptide comprises an amino acid sequence having about 80% sequence identity to SEQ ID NO: 2 to about 100% sequence identity to SEQ ID NO: 2. In some embodiments, the sFasL polypeptide comprises an amino acid sequence having about 80% sequence identity to SEQ ID NO: 2 to about 85% sequence identity to SEQ ID NO: 2, about 80% sequence identity to SEQ ID NO: 2 to about 90% sequence identity to SEQ ID NO: 2, about 80% sequence identity to SEQ ID NO: 2 to about 95% sequence identity to SEQ ID NO: 2, about 80% sequence identity to SEQ ID NO: 2 to about 96% sequence identity to SEQ ID NO: 2, about 80% sequence identity to SEQ ID NO: 2 to about 97% sequence identity to SEQ ID NO: 2, about 80% sequence identity to SEQ ID NO: 2 to about 98% sequence identity to SEQ ID NO: 2, about 80% sequence identity to SEQ ID NO: 2 to about 99% sequence identity to SEQ ID NO: 2, about 80% sequence identity to SEQ ID NO: 2 to about 100% sequence identity to SEQ ID NO: 2, about 85% sequence identity to SEQ ID NO: 2 to about 90% sequence identity to SEQ ID NO: 2, about 85% sequence identity to SEQ ID NO: 2 to about 95% sequence identity to SEQ ID NO: 2, about 85% sequence identity to SEQ ID NO: 2 to about 96% sequence identity to SEQ ID NO: 2, about 85% sequence identity to SEQ ID NO: 2 to about 97% sequence identity to SEQ ID NO: 2, about 85% sequence identity to SEQ ID NO: 2 to about 98% sequence identity to SEQ ID NO: 2, about 85% sequence identity to SEQ ID NO: 2 to about 99% sequence identity to SEQ ID NO: 2, about 85% sequence identity to SEQ ID NO: 2 to about 100% sequence identity to SEQ ID NO: 2, about 90% sequence identity to SEQ ID NO: 2 to about 95% sequence identity to SEQ ID NO: 2, about 90% sequence identity to SEQ ID NO: 2 to about 96% sequence identity to SEQ ID NO: 2, about 90% sequence identity to SEQ ID NO: 2 to about 97% sequence identity to SEQ ID NO: 2, about 90% sequence identity to SEQ ID NO: 2 to about 98% sequence identity to SEQ ID NO: 2, about 90% sequence identity to SEQ ID NO: 2 to about 99% sequence identity to SEQ ID NO: 2, about 90% sequence identity to SEQ ID NO: 2 to about 100% sequence identity to SEQ ID NO: 2, about 95% sequence identity to SEQ ID NO: 2 to about 96% sequence identity to SEQ ID NO: 2, about 95% sequence identity to SEQ ID NO: 2 to about 97% sequence identity to SEQ ID NO: 2, about 95% sequence identity to SEQ ID NO: 2 to about 98% sequence identity to SEQ ID NO: 2, about 95% sequence identity to SEQ ID NO: 2 to about 99% sequence identity to SEQ ID NO: 2, about 95% sequence identity to SEQ ID NO: 2 to about 100% sequence identity to SEQ ID NO: 2, about 96% sequence identity to SEQ ID NO: 2 to about 97% sequence identity to SEQ ID NO: 2, about 96% sequence identity to SEQ ID NO: 2 to about 98% sequence identity to SEQ ID NO: 2, about 96% sequence identity to SEQ ID NO: 2 to about 99% sequence identity to SEQ ID NO: 2, about 96% sequence identity to SEQ ID NO: 2 to about 100% sequence identity to SEQ ID NO: 2, about 97% sequence identity to SEQ ID NO: 2 to about 98% sequence identity to SEQ ID NO: 2, about 97% sequence identity to SEQ ID NO: 2 to about 99% sequence identity to SEQ ID NO: 2, about 97% sequence identity to SEQ ID NO: 2 to about 100% sequence identity to SEQ ID NO: 2, about 98% sequence identity to SEQ ID NO: 2 to about 99% sequence identity to SEQ ID NO: 2, about 98% sequence identity to SEQ ID NO: 2 to about 100% sequence identity to SEQ ID NO: 2, or about 99% sequence identity to SEQ ID NO: 2 to about 100% sequence identity to SEQ ID NO: 2. In some embodiments, the sFasL polypeptide comprises an amino acid sequence having about 80% sequence identity to SEQ ID NO: 2, about 85% sequence identity to SEQ ID NO: 2, about 90% sequence identity to SEQ ID NO: 2, about 95% sequence identity to SEQ ID NO: 2, about 96% sequence identity to SEQ ID NO: 2, about 97% sequence identity to SEQ ID NO: 2, about 98% sequence identity to SEQ ID NO: 2, about 99% sequence identity to SEQ ID NO: 2, or about 100% sequence identity to SEQ ID NO: 2. In some embodiments, the sFasL polypeptide comprises an amino acid sequence having at least about 80% sequence identity to SEQ ID NO: 2, about 85% sequence identity to SEQ ID NO: 2, about 90% sequence identity to SEQ ID NO: 2, about 95% sequence identity to SEQ ID NO: 2, about 96% sequence identity to SEQ ID NO: 2, about 97% sequence identity to SEQ ID NO: 2, about 98% sequence identity to SEQ ID NO: 2, or about 99% sequence identity to SEQ ID NO: 2. In some embodiments, the sFasL polypeptide comprises an amino acid sequence having at most about 85% sequence identity to SEQ ID NO: 2, about 90% sequence identity to SEQ ID NO: 2, about 95% sequence identity to SEQ ID NO: 2, about 96% sequence identity to SEQ ID NO: 2, about 97% sequence identity to SEQ ID NO: 2, about 98% sequence identity to SEQ ID NO: 2, about 99% sequence identity to SEQ ID NO: 2, or about 100% sequence identity to SEQ ID NO: 2. The Examples described herein provide an assay for determining whether a sFasL sequence is suitable for use (i.e., functional).

The determination of percent identity or percent similarity between two sequences can be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. Alternatively, PSI-Blast can be used to perform an iterated search which detects distant relationships between molecules. When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Alternatively, sequence alignment can be carried out using the CLUSTAL algorithm (e.g., as provided in the program Clustal-omega), as described by Higgins et al., 1996, Methods Enzymol. 266:383-402.

A polypeptide generally includes and/or refers to any of various natural or synthetic compounds containing two or more amino acids joined by a peptide bond that link the carboxyl group of one amino acid to the amino group of another. As also used herein, amino acid refers to and/or includes naturally occurring amino acids, unnatural amino acids, amino acid analogues and amino acid mimetics that function in a manner similar to a naturally occurring amino acids. Amino acids are generally referred to herein by either their name, the commonly known three letter symbols, or by the one-letter symbols recommended by the IUPAC-IUB Biochemical Nomenclature Commission.

In certain embodiments, the sFasL polypeptide comprises one or more naturally occurring amino acids. In certain embodiments, the sFasL polypeptide consists of naturally occurring amino acids. As used herein, naturally occurring amino acids include and/or refer to amino acids which are generally found in nature and are not manipulated by man. In certain instances, naturally occurring includes and/or further refers to the 20 conventional amino acids: alanine (A or Ala), cysteine (C or Cys), aspartic acid (D or Asp), glutamic acid (E or Glu), phenylalanine (F or Phe), glycine (G or Gly), histidine (H or His), isoleucine (I or Ile), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr).

In some embodiments, a sFasL polypeptide includes a functional sFasL variant of a sFasL polypeptide comprising the amino acid sequence of SEQ ID NO: 2. The Examples described herein provide an assay for determining whether a variant sFasL sequence is functional. In some embodiments, the functional sFasL variant comprises one or more mutations relative to SEQ ID NO: 2. In some embodiments, the one or more mutations comprises one or more amino acid substitutions. In certain instances, amino acid substitutions can be made in the sequence of any of the polypeptides described herein, without necessarily decreasing or ablating its activity. Accordingly, in some embodiments, the variant sequence comprises one or more amino acid substitutions. In certain embodiments, the variant sequence comprises one amino acid substitution. In certain embodiments, the variant sequence comprises two amino acid substitutions. In certain embodiments, the variant sequence comprises three amino acid substitutions. In certain instances, substitutions include conservative substitutions (e.g., substitutions with amino acids of comparable chemical characteristics). In certain instances, a non-polar amino acid can be substituted and replaced with another non-polar amino acid, wherein non-polar amino acids include alanine, leucine, isoleucine, valine, glycine, proline, phenylalanine, tryptophan and methionine. In certain instances, a neutrally charged polar amino acids can be substituted and replaced with another neutrally charged polar amino acid, wherein neutrally charged polar amino acids include serine, threonine, cysteine, tyrosine, asparagine, and glutamine. In certain instances, a positively charged amino acid can be substituted and replaced with another positively charged amino acid, wherein positively charged amino acids include arginine, lysine and histidine. In certain instances, a negatively charged amino acid can be substituted and replaced with another negatively charged amino acid, wherein negatively charged amino acids include aspartic acid and glutamic acid

In some embodiments, the nucleic acid sequence encoding the sFasL polypeptide comprises about 80% sequence identity to SEQ ID NO: 3 to about 100% sequence identity to SEQ ID NO: 3. In some embodiments, the nucleic acid sequence encoding the sFasL polypeptide comprises about 80% sequence identity to SEQ ID NO: 3 to about 85% sequence identity to SEQ ID NO: 3, about 80% sequence identity to SEQ ID NO: 3 to about 90% sequence identity to SEQ ID NO: 3, about 80% sequence identity to SEQ ID NO: 3 to about 95% sequence identity to SEQ ID NO: 3, about 80% sequence identity to SEQ ID NO: 3 to about 96% sequence identity to SEQ ID NO: 3, about 80% sequence identity to SEQ ID NO: 3 to about 97% sequence identity to SEQ ID NO: 3, about 80% sequence identity to SEQ ID NO: 3 to about 98% sequence identity to SEQ ID NO: 3, about 80% sequence identity to SEQ ID NO: 3 to about 99% sequence identity to SEQ ID NO: 3, about 80% sequence identity to SEQ ID NO: 3 to about 100% sequence identity to SEQ ID NO: 3, about 85% sequence identity to SEQ ID NO: 3 to about 90% sequence identity to SEQ ID NO: 3, about 85% sequence identity to SEQ ID NO: 3 to about 95% sequence identity to SEQ ID NO: 3, about 85% sequence identity to SEQ ID NO: 3 to about 96% sequence identity to SEQ ID NO: 3, about 85% sequence identity to SEQ ID NO: 3 to about 97% sequence identity to SEQ ID NO: 3, about 85% sequence identity to SEQ ID NO: 3 to about 98% sequence identity to SEQ ID NO: 3, about 85% sequence identity to SEQ ID NO: 3 to about 99% sequence identity to SEQ ID NO: 3, about 85% sequence identity to SEQ ID NO: 3 to about 100% sequence identity to SEQ ID NO: 3, about 90% sequence identity to SEQ ID NO: 3 to about 95% sequence identity to SEQ ID NO: 3, about 90% sequence identity to SEQ ID NO: 3 to about 96% sequence identity to SEQ ID NO: 3, about 90% sequence identity to SEQ ID NO: 3 to about 97% sequence identity to SEQ ID NO: 3, about 90% sequence identity to SEQ ID NO: 3 to about 98% sequence identity to SEQ ID NO: 3, about 90% sequence identity to SEQ ID NO: 3 to about 99% sequence identity to SEQ ID NO: 3, about 90% sequence identity to SEQ ID NO: 3 to about 100% sequence identity to SEQ ID NO: 3, about 95% sequence identity to SEQ ID NO: 3 to about 96% sequence identity to SEQ ID NO: 3, about 95% sequence identity to SEQ ID NO: 3 to about 97% sequence identity to SEQ ID NO: 3, about 95% sequence identity to SEQ ID NO: 3 to about 98% sequence identity to SEQ ID NO: 3, about 95% sequence identity to SEQ ID NO: 3 to about 99% sequence identity to SEQ ID NO: 3, about 95% sequence identity to SEQ ID NO: 3 to about 100% sequence identity to SEQ ID NO: 3, about 96% sequence identity to SEQ ID NO: 3 to about 97% sequence identity to SEQ ID NO: 3, about 96% sequence identity to SEQ ID NO: 3 to about 98% sequence identity to SEQ ID NO: 3, about 96% sequence identity to SEQ ID NO: 3 to about 99% sequence identity to SEQ ID NO: 3, about 96% sequence identity to SEQ ID NO: 3 to about 100% sequence identity to SEQ ID NO: 3, about 97% sequence identity to SEQ ID NO: 3 to about 98% sequence identity to SEQ ID NO: 3, about 97% sequence identity to SEQ ID NO: 3 to about 99% sequence identity to SEQ ID NO: 3, about 97% sequence identity to SEQ ID NO: 3 to about 100% sequence identity to SEQ ID NO: 3, about 98% sequence identity to SEQ ID NO: 3 to about 99% sequence identity to SEQ ID NO: 3, about 98% sequence identity to SEQ ID NO: 3 to about 100% sequence identity to SEQ ID NO: 3, or about 99% sequence identity to SEQ ID NO: 3 to about 100% sequence identity to SEQ ID NO: 3. In some embodiments, the nucleic acid sequence encoding the sFasL polypeptide comprises about 80% sequence identity to SEQ ID NO: 3, about 85% sequence identity to SEQ ID NO: 3, about 90% sequence identity to SEQ ID NO: 3, about 95% sequence identity to SEQ ID NO: 3, about 96% sequence identity to SEQ ID NO: 3, about 97% sequence identity to SEQ ID NO: 3, about 98% sequence identity to SEQ ID NO: 3, about 99% sequence identity to SEQ ID NO: 3, or about 100% sequence identity to SEQ ID NO: 3. In some embodiments, the nucleic acid sequence encoding the sFasL polypeptide comprises at least about 80% sequence identity to SEQ ID NO: 3, about 85% sequence identity to SEQ ID NO: 3, about 90% sequence identity to SEQ ID NO: 3, about 95% sequence identity to SEQ ID NO: 3, about 96% sequence identity to SEQ ID NO: 3, about 97% sequence identity to SEQ ID NO: 3, about 98% sequence identity to SEQ ID NO: 3, or about 99% sequence identity to SEQ ID NO: 3.

In some embodiments, the nucleic acid molecule (e.g., the vector) comprises a regulatory element that drives gene expression of the nucleic acid sequence encoding a sFasL polypeptide in a target cell (e.g., a cell within the retina). In some embodiments, the regulatory element is a promoter. In some embodiments, the promoter is a cell type-specific promoter, a tissue-specific promoter, cell-specific promoter, a ubiquitous promoter, or a response element. In certain instances, the promoter refers to a recognition site of a polynucleotide (DNA or RNA) to which an RNA polymerase binds, wherein an RNA polymerase initiates and transcribes polynucleotides linked to the promoter. In some embodiments, promoters for use in mammalian cells comprise an AT-rich region located approximately 25 to 30 bases upstream from the site where transcription is initiated and/or another sequence found 70 to 80 bases upstream from the start of transcription, a CNCAAT region where N can be any nucleotide. In certain instances, the promoter regions further comprise an enhancer, wherein an enhancer refers to a segment of DNA which contains sequences capable of providing enhanced transcription and, in some instances, can function independent of their orientation relative to another control sequence. An enhancer can function cooperatively or additively with promoters and/or other elements. In certain instances, promoter/enhancer refers to a segment of DNA which contains sequences capable of providing both promoter and enhancer functions. In certain instances, the promoter comprises or consists of a constitutive expression control sequence, wherein the constitutive expression control sequence refers to a promoter, enhancer, or promoter/enhancer that continually or continuously allows for transcription of an operably linked sequence. In some embodiments, a constitutive expression control sequence is a ubiquitous promoter, enhancer, or promoter/enhancer that allows expression in a wide variety of cell and/or tissue types. In some embodiments, the promoter is a cell or tissue-specific promoter or promoter/enhancer that allows expression in a restricted type of cell and/or tissue. In some embodiments, ubiquitous promoter sequences suitable for use in particular embodiments of the described include, but are not limited to, a cytomegalovirus (CMV), a simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and promoters from vaccinia virus, an elongation factor 1α (EFlα) promoter, early growth response 1 (EGR1), ferritin H (FerH), ferritin L (FerL), glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, and a MND promoter (a synthetic promoter that contains the U3 region of a modified MoMLV LTR with myeloproliferative sarcoma virus enhancer). In some embodiments, suitable response elements include cAMP response element, B recognition element, AhR-, dioxin- or xenobiotic-responsive element, hypoxia-responsive element, hormone response elements, serum response element, retinoic acid response elements, peroxisome proliferator hormone response elements, metal-responsive element, DNA damage response element, IFN-stimulated response elements, ROR-response element, glucocorticoid response element, calcium-response element, antioxidant response element, p53 response element, thyroid hormone response element, growth hormone response element, sterol response element, polycomb response elements, vitamin D response element, rev response element, tetracycline response element, and stress response element.

In some embodiments, the nucleic acid molecule comprising a sequence encoding a sFasL polypeptide further comprises one or more additional sequence elements. In certain embodiments, the one or more additional sequence elements comprises inverted terminal repeat (ITR) sequences, a polyadenylation signal, a stuffer sequence, or a combination thereof.

In some embodiments, the nucleic acid molecule is an expression vector. In some embodiments, the expression vector is a viral expression vector (e.g., a vector derived from a virus nucleic acid or a vector comprising viral elements).

A nucleic acid vector generally includes and/or refers to a nucleic acid molecule capable transferring or transporting another nucleic acid molecule (e.g., a nucleic acid molecule comprising a nucleic acid sequence encoding a sFasL polypeptide). The transferred nucleic acid is generally linked to (e.g., inserted into) the vector nucleic acid molecule. As described herein, a vector can include sequences that direct autonomous replication in a cell, or may include sequences sufficient to allow integration into host cell DNA. Useful vectors include, for example, plasmids (e.g., DNA plasmids or R A plasmids), transposons, cosmids, bacterial artificial chromosomes, and viral vectors.

A viral nucleic acid vector generally includes and/or refers to a nucleic acid molecule (e.g., a nucleic acid molecule comprising a nucleic acid sequence encoding a sFasL polypeptide) that includes virus-derived nucleic acid elements that facilitate packaging of the nucleic acid molecule into a viral particle and/or the transfer of the nucleic acid molecule, or a sequence thereof. Viral particles will typically include various viral components and sometimes also host cell components in addition to nucleic acid(s). In some instances, a viral vector includes and/or refers to the viral nucleic acid vector (e.g., a nucleic acid molecule comprising virus derived nucleic acid elements and a nucleic acid sequence encoding a sFasL polypeptide) and the viral particle (e.g., a capsid protein). In some instances, a viral vector includes and/or refers to a viral particle (e.g., a capsid protein) comprising the viral nucleic acid vector (e.g., a nucleic acid molecule comprising virus derived nucleic acid elements and a nucleic acid sequence encoding a sFasL polypeptide).

In some embodiments, the viral vector is the viral vector is selected from the group consisting of: an adenovirus vector, an adeno-associated virus (AAV) vector, a lentivirus vector, a herpesvirus vector, a poxvirus vector, a baculovirus vector, a papillomavirus vector, and a papovavirus vector. In some embodiments, the viral vector comprises an adenovirus vector. In some embodiments, the viral vector comprises an adeno-associated virus vector. In some embodiments, the viral vector comprises a lentivirus vector. In some embodiments, the viral vector comprises a herpesvirus vector. In some embodiments, the viral vector comprises a poxvirus vector. In some embodiments, the viral vector comprises a baculovirus vector. In some embodiments, the viral vector comprises a papillomavirus vector. In some embodiments, the viral vector comprises a papovavirus vector

In some embodiments, the viral vector is an AAV vector. An AAV generally includes and refers to an isolated AAV that has been artificially produced or obtained. Isolated AAVs can be produced using recombinant methods. Such AAVs are generally referred to as recombinant AAVs. Recombinant AAVs (rAAVs) can have tissue-specific targeting capabilities, such that a transgene (e.g., a nucleic acid sequence encoding a sFasL polypeptide) of the rAAV is delivered specifically to one or more predetermined tissue(s). The AAV capsid generally tissue-specific targeting and/or or a cell specific promoter capabilities. Thus, an rAAV having a capsid appropriate for the tissue being targeted can be selected for targeting cells within the retina (e.g., photoreceptors, retinal pigment epithelial cells, retinal ganglion cells, or a combination thereof).

Methods for obtaining recombinant AAVs having a desired capsid protein are well known in the art. Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2, AAV3, AAV4, AAV5, AAV6, AAV8, AAVrh8, AAV9, AAV10, AAV11, AAV12, and AAV13. In some embodiments, an AAV capsid protein is of a serotype derived from a non-human primate, for example AAVrh8 serotype. In some embodiments, the AAV capsid protein is of a serotype that has tropism for the eye of a subject, for example an AAV (e.g., AAV2, AAV5, AAV6, AAV6.2, AAV7, AAV8, AAV9, AAVrh.8, AAVrh. 10, AAVrh.39 and AAVrh.43) that transduces ocular cells of a subject more efficiently than other vectors. In some embodiments, an AAV capsid protein comprises an AAV2 capsid sequence, AAV5 capsid sequence, AAV8 capsid sequence, or a AAV9 capsid sequence. In some embodiments, the AAV capsid protein comprises the sequence set forth in SEQ ID NO: 4.

The components to be cultured in the host cell to package a rAAV vector in an AAV capsid can be provided to the host cell in trans. Alternatively, any one or more of the required components (e.g., recombinant AAV vector, rep sequences, cap sequences, and/or helper functions) can be provided by a stable host cell which has been engineered to contain one or more of the required components using methods known to those of skill in the art. Most suitably, such a stable host cell will contain the required component(s) under the control of an inducible promoter. However, the required component(s) can be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell can be generated which is derived from 293 cells (which contain E1 helper functions under the control of a constitutive promoter), but which contain the rep and/or cap proteins under the control of inducible promoters. Still other stable host cells can be generated by one of skill in the art.

The recombinant AAV vector, rep sequences, cap sequences, and helper functions required for producing the rAAV of the disclosure can be delivered to the packaging host cell using any appropriate genetic element (vector). The selected genetic element can be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs can be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Transfection generally includes and refers to the uptake of foreign/exogenous DNA by a cell, and a cell has been transfected when exogenous DNA has been introduced inside the cell membrane.

A host cell generally includes and refers to any cell that harbors, or is capable of harboring, a substance of interest. Often a host cell is a mammalian cell. A host cell can be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein can refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

An effective amount of AAV can depend on the rAAV used. The compositions and methods described herein is based, in part, on the recognition that certain rAAVs comprising capsid proteins mediate efficient transduction of ocular (e.g., photoreceptor, retinal, etc.) cells. In some embodiments, an rAAV used in methods provided herein comprises a capsid protein of an AAV serotype selected from the group consisting of: AAV2, AAV5, AAV6, AAV6.2, AAV7, AAV8, AAV9, AAVrh.8, AAVrh. 10, AAVrh.39, and AAVrh.43. In some embodiments, the rAAV comprises a capsid protein of AAV2 serotype (SEQ ID NO: 4). In some embodiments, the capsid protein comprises an amino acid sequence that is at least 70%, at least 80%, at least 90%, at least 95%, or at least 99% identical to SEQ ID NO: 4. In some embodiments, the capsid protein is AAV2 capsid protein.

In certain embodiments, the effective amount of rAAV is 10¹⁰, 10¹¹, 10¹², 10¹³, or 10¹⁴ genome copies per kg. In certain embodiments, the effective amount of rAAV is 10¹⁰, 10¹¹, 10¹², 10¹³, 10¹⁴, or 10¹⁵ genome copies per subject.

An effective amount can also depend on the mode of administration. For example, targeting an ocular (e.g., photoreceptor, retinal, etc.) tissue by intrastromal administration or subcutaneous injection can require different (e.g., higher or lower) doses, in some cases, than targeting an ocular (e.g., photoreceptor, retinal, etc.) tissue by another method (e.g., systemic administration, topical administration). In some embodiments, intrastromal injection (IS) of rAAV having certain serotypes (e.g., AAV2, AAV5, AAV6, AAV6.2, AAV7, AAV8, AAV9, AAVrh.8, AAVrh.10, AAVrh.39, and AAVrh.43) mediates efficient transduction of ocular (e.g., corneal, photoreceptor, retinal, etc.) cells. Thus, in some embodiments, the injection is intrastromal injection (IS). In some embodiments, the administration is via injection, optionally subretinal injection or intravitreal injection. In some embodiments, the injection is topical administration (e.g., topical administration to an eye). In some cases, multiple doses of a rAAV are administered. In some embodiments, the composition is administered via intravitreal injection, intracameral injection, injection into the suprachoroidal space, injection in the subtenons space, subconjunctival injection, retrobulbar injection, peribulbar injection, microneedle injection, subretinal injection, or subretinal infusion.

In some embodiments, efficient transduction of ocular (e.g., photoreceptor, retinal, etc.) cells by rAAV described herein can be useful for the treatment of a subject having retinal epithelial cell death (e.g., as in macular degeneration). In some embodiments, administration of a rAAV (or isolated nucleic acid) as described by the disclosure results in transduction of a retinal neuron. Examples of retinal neurons include, but are not limited to, bipolar cells, ganglion cells, horizontal cells, retina amacrine cells, rod cells and cone cells. In some embodiments, administration of a rAAV (or isolated nucleic acid) as described by the disclosure results in transduction of a retinal pigment epithelial (RPE) and/or a photoreceptor.

### Methods of Treating Retinal Pigment Epithelial (RPE) Cell Death

Provided herein are methods useful for treating RPE cell death, photoreceptor cell death associated with or resulting from RPE cell death, and/or diseases associated with or resulting from RPE cell death. In certain instances, treating RPE cell death (e.g., inhibiting RPE cell death) provided the platform for treating photoreceptor death associated with or resulting from RPE cell death. As described herein, the methods useful for treating RPE cell death, photoreceptor cell death associated with or resulting from RPE cell death, and/or diseases associated with or resulting from RPE cell death comprises increasing an amount of a sFasL polypeptide within the eye of an individual (e.g., within retinal tissue).

Provided and described herein are methods of treating retinal pigment epithelial (RPE) cell death in an eye of an individual, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for the treatment of retinal pigment epithelial (RPE) cell death.

In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof or status (e.g., a detectable physiological state) associated therewith comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof.

As described, the methods and compositions described herein are useful for preventing, inhibiting, and/or managing macular degeneration and related indications, such as, but not limited to, atrophic (dry) MD, exudative (wet) MD, age-related maculopathy (ARM), and atrophy of retinal pigment epithelium (RPE).

Macular degeneration (MD) generally refers to and encompasses all forms of macular degenerative diseases regardless of a patient's age, although some macular degenerative diseases are more common in certain age groups. Symptoms associated with MD and related syndromes include, but are not limited to, drusen rounded whitish-yellowish spots in the fundus, submacular disciform scar tissue, choroidal neovascularisation, retinal pigment epithelium detachment, atrophy of retinal pigment epithelium, abnormal blood vessels stemming from the choroid (the blood vessel-rich tissue layer just beneath the retina), a blurry or distorted area of vision, a central blind spot, pigmentary abnormalities, a continuous layer of fine granular material deposited in the inner part of Bruch's membrane, and a thickening and decreased permeability of Bruch's membrane. Causes of MD include, but are not limited to, genetic, physical trauma, diseases such as diabetes, and infection, such as bacterial infection (e.g., leprosy and ENL in particular). The compounds of the invention can effectively treat, prevent or manage all types of MD and related syndromes or symptoms, regardless of their causes.

In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy. In some embodiments, the disorder is retinitis pigmentosa. In some embodiments, the disorder is Stargardt's disease.

Further provided and described herein are methods of treating photoreceptor cell death in an eye of an individual having retinal pigment epithelial (RPE) cell death, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for the treatment of retinal pigment epithelial (RPE) cell death for treating photoreceptor cell death in an eye of an individual having retinal pigment epithelial (RPE) cell death. In some embodiments, photoreceptor cell death comprises a decrease in electrical activity of photoreceptor cells (e.g., as measured by electroretinogram), a decrease in photoreceptor density (e.g., as measured by adaptive optics scanning laser ophthalmoscopy), or a combination thereof.

In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof or status (e.g., a detectable physiological state) associated therewith comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof.

In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy. In some embodiments, the disorder is retinitis pigmentosa. In some embodiments, the disorder is Stargardt's disease.

Provided and described herein are methods of treating macular degeneration in an eye of an individual, the method comprising: administering to the eye a nucleic acid molecule comprising a nucleic acid sequence encoding a soluble Fas ligand (sFasL) polypeptide. Also provided herein is the use of a nucleic acid molecule comprising a nucleic acid sequence encoding a soluble Fas ligand (sFasL) polypeptide for the treatment of macular degeneration.

In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy.

Provided and described herein are methods of treating retinitis pigmentosa in an eye of an individual, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for treating retinitis pigmentosa in an eye of an individual.

Provided and described herein are methods of treating Stargardt's disease in an eye of an individual, the method comprising: administering to the eye a nucleic acid molecule comprising a nucleic acid sequence encoding a soluble Fas ligand (sFasL) polypeptide. Also provided is the use of a nucleic acid molecule comprising a nucleic acid sequence encoding a soluble Fas ligand (sFasL) polypeptide for treating Stargardt's disease in an eye of an individual.

In some embodiments, the sFasL polypeptide comprises a Fas ligand (FasL) polypeptide comprising (i) an amino acid sequence having at least 90% sequence identity to SEQ ID NO: 1 and (ii) a truncation at an N-terminus of the FasL polypeptide. In some embodiments, the sFasL polypeptide comprises (i) an amino acid sequence having at least 90% sequence identity to SEQ ID NO: 1 and (ii) a truncation at an N-terminus of the FasL polypeptide. In some embodiments, the truncation comprises a deletion of amino acids 1 to about 130, amino acids 1 to about 125, amino acids 1 to about 120, or amino acids 1 to about 115 from SEQ ID NO: 1. In some embodiments, the sFasL polypeptide comprises an amino acid sequence at least 90% sequence identity to SEQ ID NO: 2. In some embodiments, the sFasL polypeptide comprises SEQ ID NO: 2. In some embodiments, the sFasL polypeptide consists of an amino acid sequence as set forth in SEQ ID NO: 2.

In some embodiments, the nucleic acid sequence comprises a sequence identity of at least 90% to SEQ ID NO: 3 and encodes the sFasL polypeptide comprising (i) SEQ ID NO: 2 or (ii) an amino acid sequence at least 90% sequence identity to SEQ ID NO: 2. In some embodiments, the nucleic acid sequence comprises SEQ ID NO: 3. In some embodiments, the nucleic acid sequence is configured for expression in one or more cells in the eye of the individual. In some embodiments, the nucleic acid molecule comprises a promoter comprising a promoter sequence, and wherein the promoter sequence is operably linked to the nucleic acid sequence encoding the soluble sFasL polypeptide. In some embodiments, the promoter is a ubiquitous promoter. In some embodiments, the promoter is a tissue-specific promoter. In some embodiments, the promoter promotes expression of a transgene in one or more cells within the retina (e.g., retinal pigment epithelial cells, retinal ganglion cells, photoreceptors, immune cells, etc.).

In some embodiments, the nucleic acid molecule is a nucleic acid expression vector. In some embodiments, the nucleic acid expression vector is a viral vector. In some embodiments, the viral vector is selected from the group consisting of: an adenovirus vector, an adeno-associated virus vector, a lentivirus vector, a herpesvirus vector, a poxvirus vector, a baculovirus vector, a papillomavirus vector, and a papovavirus vector. In some embodiments, the viral vector comprises an adenovirus vector. In some embodiments, the viral vector comprises an adeno-associated virus vector. In some embodiments, the viral vector comprises a lentivirus vector. In some embodiments, the viral vector comprises a herpesvirus vector. In some embodiments, the viral vector comprises a poxvirus vector. In some embodiments, the viral vector comprises a baculovirus vector. In some embodiments, the viral vector comprises a papillomavirus vector. In some embodiments, the viral vector comprises a papovavirus vector. In some embodiment the viral vector comprises an AAV2, AAV5, AAV8, AAV9 viral vector or a chimera thereof.

In some embodiments, a viral particle or viral-like particle comprising the viral vector is administered to the eye. In some embodiments, the viral-like particle is an AAV capsid protein. In some embodiments, the AAV capsid protein comprises an AAV2 capsid sequence, an AAV5 capsid sequence, an AAV8 capsid sequence, and or an AAV9 capsid sequence. In some embodiments, the nucleic acid molecule (e.g., the viral vector) is administered to the vitreous of the eye.

Further provided and descried herein are methods of treating retinal pigment epithelial (RPE) cell death or a disorder associated therewith in an eye of an individual, the method comprising: administering a sFasL therapeutic (e.g., a sFasL polypeptide or nucleic acid configured to express sFasL) to the eye, wherein the sFasL therapeutic increases an amount of sFasL polypeptide in the eye. In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof or status (e.g., a detectable physiological state) associated therewith comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof. In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy. In some embodiments, the disorder is retinitis pigmentosa. In some embodiments, the disorder is Stargardt's disease.

Provided and described herein are methods of treating photoreceptor cell death in an eye of an individual having retinal pigment epithelial (RPE) cell death, the method comprising: administering a sFasL therapeutic to the eye, wherein the sFasL therapeutic increases an amount of sFasL polypeptide in the eye. In some embodiments, the RPE cell death comprises a reduction and/or decrease in a number of RPE cells within the eye. In some embodiments, the eye has retinal pigment epithelial (RPE) cell death or a symptom thereof. In some embodiments, the symptom thereof or status (e.g., a detectable physiological state) associated therewith comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof. In some embodiments, the individual has an ocular disease or disorder. In some embodiments, the disorder is macular degeneration. In some embodiments, the macular degeneration is acute macular degeneration. In some embodiments, the macular degeneration comprises chronic macular degeneration. In some embodiments, the macular degeneration comprises wet macular degeneration. In some embodiments, the macular degeneration comprises dry macular degeneration. In some embodiments, the macular degeneration comprises age-related macular degeneration. In certain embodiments, the age-related macular degeneration comprises wet macular degeneration or dry macular degeneration. In some embodiments, the macular degeneration comprises or is characterized by geographic atrophy. In some embodiments, the disorder is retinitis pigmentosa. In some embodiments, the disorder is Stargardt's disease.

In some embodiments, the sFasL polypeptide comprises SEQ ID NO: 3. In some embodiments, the sFasL therapeutic comprises the sFasL polypeptide. In some embodiments, the sFasL therapeutic comprises a nucleic acid molecule encoding the sFasL polypeptide. In some embodiments, the sFasL therapeutic comprises a composition comprising a nucleic acid molecule encoding the sFasL polypeptide and carrier comprising the nucleic acid molecule encoding the sFasL polypeptide.

As used herein the term individual, patient, or subject refers to individuals diagnosed with, suspected of being afflicted with, or at-risk of developing at least one disease, condition, or status for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein, the terms “treatment” or “treating” are used in reference to a pharmaceutical or other intervention regimen used for obtaining beneficial or desired results in the recipient. Beneficial or desired results include but are not limited to a therapeutic benefit and/or a prophylactic benefit. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject can still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease can not have been made. Skilled artisans will recognize that given a population of potential individuals for treatment not all will respond or respond equally to the treatment. Such individuals are considered treated.

As used herein, the words comprising (and any form of comprising, such as comprise and comprises), having (and any form of having, such as have and has), including (and any form of including, such as include and includes) or containing (and any form of containing, such as contain and contains), are inclusive or open-ended and do not exclude additional, unrecited elements or process steps. As also used herein, in any instance or embodiment described herein, comprising can be replaced with consisting essentially of and/or consisting of. used herein, in any instance or embodiment described herein, comprises can be replaced with consists essentially of and/or consists of.

As used herein, the term about in the context of a given value or range includes and/or refers to a value or range that is within 20%, within 10%, and/or within 5% of the given value or range.

As used herein, the term and/or is to be taken as specific disclosure of each of the two specified features or components with or without the other. For example, A and/or B is to be taken as specific disclosure of each of (i) A, (ii) B and (iii) A and B, just as if each were set out individually herein.

As used herein, a sample includes and/or refers to any fluid or liquid sample which is being analyzed in order to detect and/or quantify an analyte. In some embodiments, a sample is a biological sample. Examples of samples include without limitation a bodily fluid, an extract, a solution containing proteins and/or DNA, a cell extract, a cell lysate, or a tissue lysate. Non-limiting examples of bodily fluids include urine, saliva, blood, serum, plasma, cerebrospinal fluid, tears, semen, sweat, pleural effusion, liquified fecal matter, and lacrimal gland secretion.

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein can be employed in practicing the invention. It is intended that the following claims define the scope of the embodiments disclosed herein, and that methods and structures within the scope of these claims and their equivalents be covered thereby.

### EXAMPLES

### Example 1: Fas Inhibition and Retinal Cell Protection

Fas is a transmembrane protein and a member of the tumor necrosis factor receptor gene superfamily. Binding of the Fas ligand (FasL) to the Fas receptor results in activation of the caspase cascade and cell death. Fas receptor activation also results in upregulation of cytokine and chemokine production, leading to a pro-inflammatory state. Fas receptor activation has been demonstrated in various models of retinal disease such as retinal detachment, oxidative stress and elevated intraocular pressure. In these models, regardless of the stressor, there is increased expression of Fas, activation of the caspase cascade—typically demonstrated by the cleavage and activation of caspase 8, and increased cell death. This is accompanied by elevated cytokine and chemokine expression and increased Iba-1 staining in the retina, with Iba-1 being a marker of microglia and macrophages. Thus, Fas activation represents a core, common pathophysiologic mechanism regulating retinal cell death across a variety of disease states.

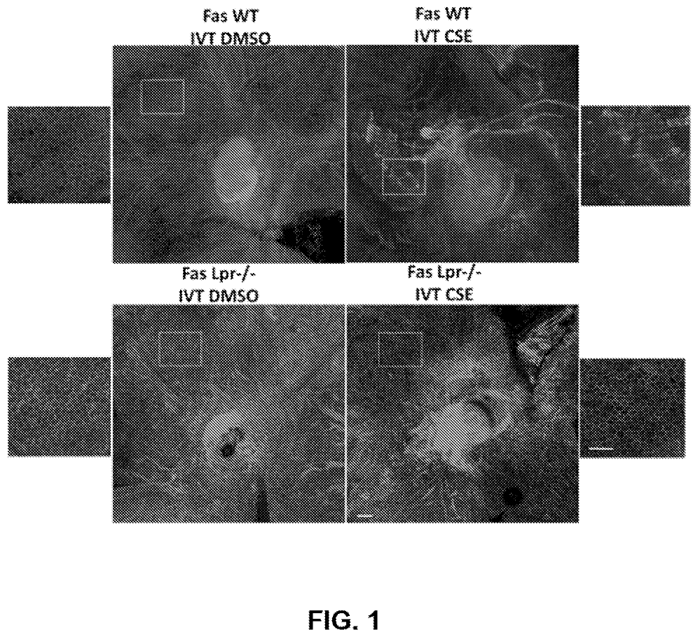

To further assess the potential role of Fas in age-related macular degeneration (AMD, a disorder characterized by RPE cell death), an acute cigarette smoke extract (CSE) model of RPE atrophy was used. Since cigarette smoking is a major risk factor for AMD formation, this acute model mirrors an aspect of the human condition. CSE is a potent oxidative stressor and was injected into the vitreous cavity of the mouse eyes to induce RPE cell death and atrophy. FIG. 1 shows intravitreal injection of CSE in wild-type mice resulted in severe damage to the normal RPE architecture, as demonstrated by the massive disruption of the normal honeycomb pattern of the ZO-1 staining.

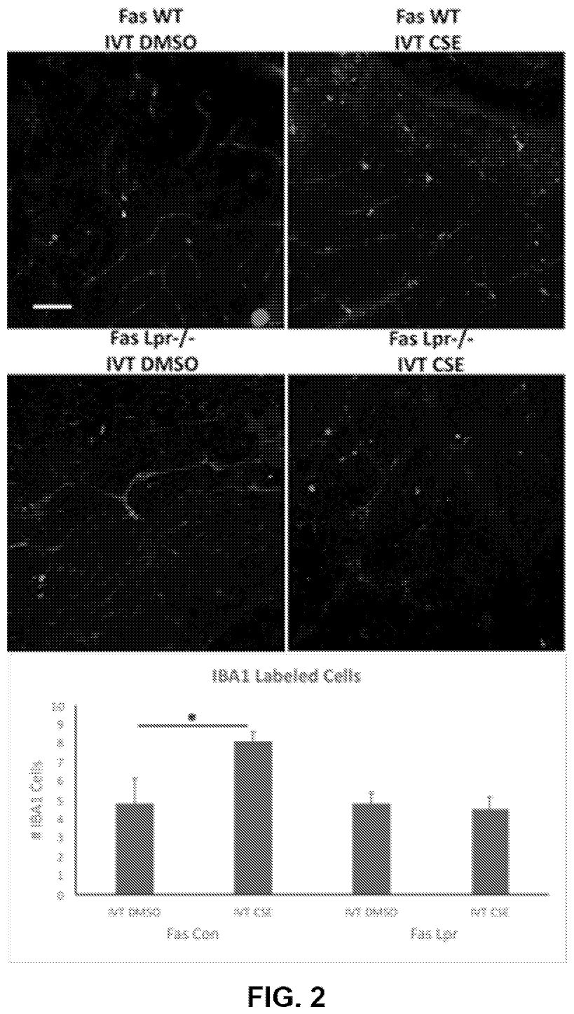

To investigate the potential role of Fas in the RPE death seen in the CSE model, the CSE model was tested in the lpr mouse. The lpr mouse contains a point mutation that renders the Fas receptor inactive, essentially creating a functional knockout of the gene. The lpr mouse has a normal RPE and retina, both in terms of structure and function. In contrast to the effect of CSE in a normal mouse eye, injection of CSE into the vitreous of the lpr mouse did not result in any major disruption of the RPE staining pattern, consistent with the protection of the RPE to this severe oxidative stress injury. FIG. 1 and FIG. 2 show that Fas receptor inactivation was able to treat and/or prevent the RPE cells after CSE injection. In this model, there was also a significant increase in Iba1-positive cells (microglia and macrophages) in the outer retina following administration of CSE to the vitreous cavity. Inhibiting Fas activation genetically, with the lpr mouse, resulted in significant reduction of the Iba1-positive staining, consistent with reduced activation of microglia and macrophages typically associated with this type of severe oxidative stress injury. FIG. 1 further shows that Fas deficiency protects the RPE from cigarette smoke extract-induced cell death. Wild-type control (Fas WT) or lpr^{-/-} (Fas Lpr−/−) mice (n=5 each) were given intravitreal CSE (250 μg/ml) in one eye or DMSO vehicle in the contralateral eye. After 10 days RPE flatmounts were immunolabeled for ZO-1 to outline cell shape. The RPE of the wild-type mice given vehicle had preserved cobblestone morphology (top left of FIG. 1) while the RPE of wild-type mice given CSE had disrupted ZO-1 labeling with ill-defined cell shape and enlarged, irregular contour (top right of FIG. 1). In contrast, the RPE of lpr^{-/-} mice given either vehicle (bottom left) or CSE (bottom right of FIG. 1) had regular cobblestone cell shape. Insets show magnified view of the RPE. Bar=50 μm.

FIG. 2 shows a reduction in the activation of microglia and macrophages within the retina upon Fas receptor inactivation. Fas deficiency reduces the inflammatory microenvironment following cigarette smoke extract. Wild-type control (Fas WT) or lpr^{-/-} (Fas Lpr−/−) mice (n=5 each) were given intravitreal CSE in one eye or vehicle in the contralateral eye. After 10 days retinal flatmounts were immunolabeled for Iba-1 to identify macrophages and microglia. Compared to control retina (upper left panel FIG. 2), the number of Iba-1 labeled cells (arrowhead) are increased after CSE stimulation (upper right panel FIG. 2), which is decreased in the retinas of lpr^{-/-} mice given either DMSO vehicle or CSE. Bar=50 μm. Graph of FIG. 2 quantifies the number of IBA1 labeled cells in the retinas. *p<0.05.

Taken together, these data demonstrate that Fas inhibition, in addition to preserving cell viability, can reduce inflammation in animal models of RPE atrophy.

### Example 2: Gene Therapy for Inhibiting Fas

While FasL binds Fas, the outcome of that interaction is dependent on the form of the bound FasL—whether membrane-bound or soluble. The difference between soluble and membrane-bound FasL has observed in glaucoma disease models, however due to the physiological differences in glaucoma pathogenesis (e.g., involving death of retinal ganglion cells (RGCs)) it was unknown as to whether FasL protection and treatment would be effective in disease physiologies characterized by RPE cell death (e.g., macular degeneration).

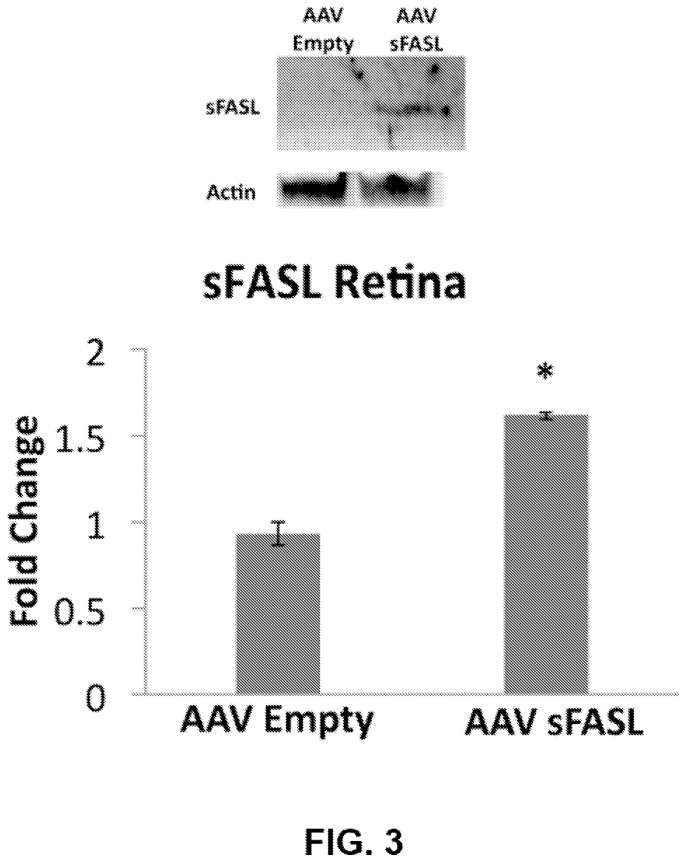

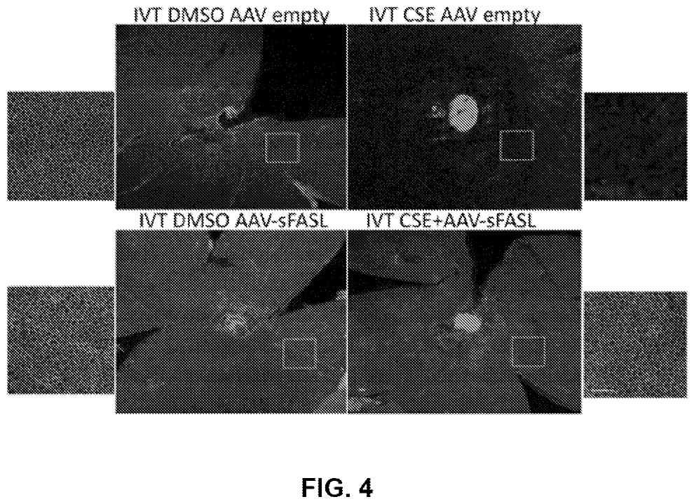

To explore the potential of Fas inhibition as a therapeutic approach for treating dry AMD (a disorder characterized by RPE cell death), a soluble Fas ligand (sFasL) gene therapy construct was used in the acute CSE mouse model. In this construct, an AAV2 vector is used to overexpress the soluble FasL (e.g., having a constitutive, ubiquitous promoter operably linked to a nucleic acid sequence encoding a sFasL polypeptide) to act as a constitutive inhibitor of Fas. FIG. 3 shows that the intravitreal injection of the AAV-sFasL vector results in a measurable increase in the protein level of sFasL detected in the retina. The true level of sFasL, though, may actually be higher, but due to the secreted nature of the protein, wherein a significant percentage of the sFasL may not have been captured by this assay. FIG. 4 shows RPE flatmounts of the control (empty vector) versus AAV-sFasL treated eyes after intravitreal injection of CSE. Fas inhibitor gene therapy AAV2-sFasL protects RPE from cigarette smoke extract-induced damage. Wild-type mice (n=5 per group) were given IVT AAV2-sFasL or IVT AAV2-empty vector in each eye. Two weeks later, mice were given IVT CSE in one eye or DMSO vehicle in the contralateral eye. After 10 days RPE flatmounts were immunolabeled with anti-ZO-1. The RPE has lost its hexagonal shape after CSE injection. This damage is mitigated by AAV2-sFasL treatment. White boxes highlight the location of the magnified images of the RPE. Bar=50 μm. As can be seen, the overexpression of sFasL resulted in marked protection of the normal RPE architecture. This protection was accompanied by a notable reduction in the CSE-induced increase in Iba1-positive cells in the retina.

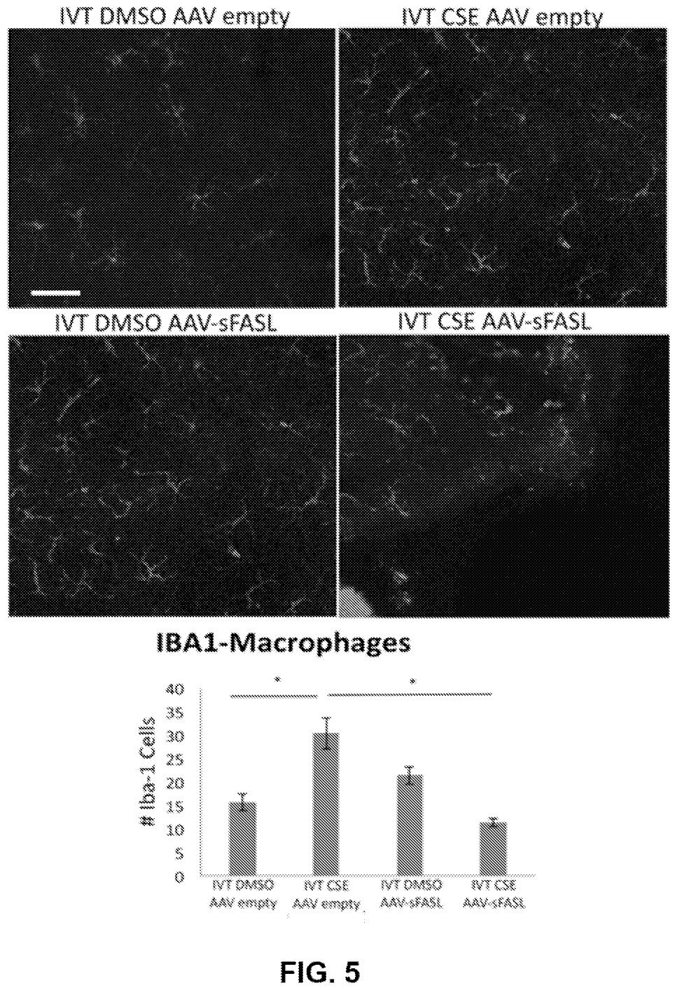

FIG. 5 shows the reduction in the CSE-induced increase in Iba1-positive cells in the retina. Fas inhibitor gene therapy AAV2-sFasL reduces the inflammatory microenvironment following cigarette smoke extract. Wild-type mice (n=5 per group) were given IVT AAV2-sFasL or IVT AAV2-empty vector in each eye. Two weeks later, mice were given IVT CSE in one eye or DMSO vehicle in the contralateral eye. After 10 days retinal flatmounts were immunolabeled for Iba-1 to identify macrophages and microglia. Bar=50 μm. Data also show that the number of Iba-1 labeled cells increases with CSE and that AAV2-sFasL decreases the number of Iba-1 labeled cells to control levels in CSE treated mice. *p<0.05. These data provide compelling evidence that blocking Fas receptor activation, genetically and pharmacologically, inhibits RPE damage in the face of acute oxidative stress.

SEQUENCES

| SEQ | | |
|---|---|---|
| ID | | |
| NO | SEQUENCE | DESCRIPTION |
| 1 | MQQPFNYPYPQIYWVDSSASSPWAPPGTVLPCPT | FasL |
| | SVPRRPGQRRPPPPPPPPPLPPPPPPPPLPPLPL | |
| | PPLKKRGNHSTGLCLLVMFFMVLVALVGLGLGMF | |
| | QLFHLQKELAELRESTSQMHTASSLEKQIGHPSP | |
| | PPEKKELRKVAHLTGKSNSRSMPLEWEDTYGIVL | |
| | LSGVKYKKGGLVINETGLYFVYSKVYFRGQSCNN | |
| | LPLSHKVYMRNSKYPQDLVMMEGKMMSYCTTGQM | |
| | WARSSYLGAVENLTSADHLYVNVSELSLVNFEES | |
| | QTFFGLYKL | |
| 2 | EKQIGHPSPPPEKKELRKVAHLTGKSNSRSMPLE | sFasL |
| | WEDTYGIVLLSGVKYKKGGLVINETGLYFVYSKV | |
| | YFRGQSCNNLPLSHKVYMRNSKYPQDLVMMEGKM | |
| | MSYCTTGQMWARSSYLGAVENLTSADHLYVNVSE | |
| | LSLVNFEESQTFFGLYKL | |
| 3 | gagaagcaaataggccaccccagtccaccccctg | sFasL |
| | aaaaaaaggagctgaggaaagtggcccatttaac | |
| | aggcaagtccaactcaaggtccatgcctctggaa | |
| | tgggaagacacctatggaattgtcctgctttctg | |
| | gagtgaagtataagaagggtggccttgtgatcaa | |
| | tgaaactgggctgtactttgtatattccaaagta | |
| | tacttccggggtcaatcttgcaacaacctgcccc | |
| | tgagccacaaggtctacatgaggaactctaagta | |
| | tccccaggatctggtgatgatggaggggaagatg | |
| | atgagctactgcactactgggcagatgtgggccc | |
| | gcagcagctacctgggggcagtgttcaatcttac | |
| | cagtgctgatcatttatatgtcaacgtatctgag | |
| | ctctctctggtcaattttgaggaatctcagacgt | |
| | ttttcggcttatataagctctaa | |
| 4 | MAADGYLPDWLEDTLSEGIRQWWKLKPGPPPPKP | AAV2 |
| | AERHKDDSRGLVLPGYKYLGPFNGLDKGEPVNEA | capsid |
| | DAAALEHDKAYDRQLDSGDNPYLKYNHADAEFQE | |
| | RLKEDTSFGGNLGRAVFQAKKRVLEPLGLVEEPV | |
| | KTAPGKKRPVEHSPVEPDSSSGTGKAGQQPARKR | |
| | LNFGQTGDADSVPDPQPLGQPPAAPSGLGTNTMA | |
| | TGSGAPMADNNEGADGVGNSSGNWHCDSTWMGDR | |
| | VITTSTRTWALPTYNNHLYKQISSQSGASNDNHY | |
| | FGYSTPWGYFDFNRFHCHFSPRDWQRLINNNWGF | |
| | RPKRLNFKLFNIQVKEVTQNDGTTTIANNLTSTV | |
| | QVFTDSEYQLPYVLGSAHQGCLPPFPADVFMVPQ | |
| | YGYLTLNNGSQAVGRSSFYCLEYFPSQMLRTGNN | |
| | FTFSYTFEDVPFHSSYAHSQSLDRLMNPLIDQYL | |
| | YYLSRTNTPSGTTTQSRLQFSQAGASDIRDQSRN | |
| | WLPGPCYRQQRVSKTSADNNNSEYSWTGATKYHL | |
| | NGRDSLVNPGPAMASHKDDEEKFFPQSGVLIFGK | |
| | QGSEKTNVDIEKVMITDEEEIRTINPVATEQYGS | |
| | VSTNLQRGNRQAATADVNTQGVLPGMVWQDRDVY | |
| | LQGPIWAKIPHTDGHFHPSPLMGGFGLKHPPPQI | |
| | LIKNTPVPANPSTTFSAAKFASFITQYSTGQVSV | |
| | EIEWELQKENSKRWNPEIQYTSNYNKSVNVDFTV | |
| | DINGVYSEPRPIGTRYLTRNL | |

1. A method of treating retinal pigment epithelial (RPE) cell death in an eye of an individual, the method comprising:
administering to the eye a nucleic acid molecule comprising a nucleic acid sequence encoding a soluble Fas ligand (sFasL) polypeptide.

2. A method of treating photoreceptor cell death in an eye of an individual having retinal pigment epithelial (RPE) cell death, the method comprising:
administering to the eye a nucleic acid molecule comprising a nucleic acid sequence encoding a soluble Fas ligand (sFasL) polypeptide.

3. The method of claim 2, wherein photoreceptor cell death comprises a decrease in electrical activity of photoreceptor cells (e.g., as measured by electroretinogram), a decrease in photoreceptor density (e.g., as measured by adaptive optics scanning laser ophthalmoscopy), or a combination thereof.

4. The method of any one of claims 1 to 3, wherein the RPE cell death comprises a reduction and/or decrease in a number of RPE cells within the eye.

5. The method of any one of claims 1 to 4, wherein the eye has retinal pigment epithelial (RPE) cell death or a symptom thereof.

6. The method of claim 5, wherein the symptom thereof comprises a presence drusen in the eye, abnormal or rupture vasculature (e.g., blood vessels) in the eye, an increased amount of cytokines in the eye (e.g., in a vitreous humor or aqueous humor sample from the eye) relative to a control eye, elevated intraocular pressure, or a combination thereof.

7. A method of treating macular degeneration in an eye of an individual, the method comprising:
administering to the eye a nucleic acid molecule comprising a nucleic acid sequence encoding a soluble Fas ligand (sFasL) polypeptide.

8. The method of claim 7, wherein the macular degeneration is acute macular degeneration.

9. The method of claim 7, wherein the macular degeneration comprises chronic macular degeneration.

10. The method of claim 7, wherein the macular degeneration comprises wet macular degeneration.

11. The method of claim 7, wherein the macular degeneration comprises dry macular degeneration.

12. The method of claim 7, wherein the macular degeneration comprises age-related macular degeneration.

13. The method of claim 12, wherein the age-related macular degeneration comprises wet macular degeneration or dry macular degeneration.

14. A method of treating retinitis pigmentosa in an eye of an individual, the method comprising:
administering to the eye a nucleic acid molecule comprising a nucleic acid sequence encoding a soluble Fas ligand (sFasL) polypeptide.

15. A method of treating Stargardt's disease in an eye of an individual, the method comprising:
administering to the eye a nucleic acid molecule comprising a nucleic acid sequence encoding a soluble Fas ligand (sFasL) polypeptide.

16. The method of any one of claims 1 to 15, wherein the sFasL polypeptide comprises a Fas ligand (FasL) polypeptide comprising (i) an amino acid sequence having at least 90% sequence identity to SEQ ID NO: 1 and (ii) a truncation at an N-terminus of the FasL polypeptide.

17. The method of any one of claims 1 to 15, wherein the sFasL polypeptide comprises (i) an amino acid sequence having at least 90% sequence identity to SEQ ID NO: 1 and (ii) a truncation at an N-terminus of the FasL polypeptide.

18. The method of any one of claims 16 to 17, wherein the truncation comprises a deletion of amino acids 1 to about 130, amino acids 1 to about 125, amino acids 1 to about 120, or amino acids 1 to about 115 from SEQ ID NO: 1.

19. The method of any one of claims 1 to 18, wherein the sFasL polypeptide comprises an amino acid sequence at least 90% sequence identity to SEQ ID NO: 2.

20. The method of any one of claims 1 to 19, wherein the sFasL polypeptide comprises SEQ ID NO: 2.

21. The method of any one of claims 1 to 20, wherein the sFasL polypeptide consists of an amino acid sequence as set forth in SEQ ID NO: 2.

22. The method of any one of claims 1 to 21, wherein the nucleic acid sequence comprises a sequence identity of at least 90% to SEQ ID NO: 3 and encodes the sFasL polypeptide comprising (i) SEQ ID NO: 2 or (ii) an amino acid sequence at least 90% sequence identity to SEQ ID NO: 2.

23. The method of any one of claims 1 to 22, wherein the nucleic acid sequence comprises SEQ ID NO: 3.

24. The method of any one of claims 1 to 23, wherein the nucleic acid sequence is configured for expression in one or more cells in the eye of the individual.

25. The method of any one of claims 1 to 24, wherein the nucleic acid molecule comprises a promoter comprising a promoter sequence, and wherein the promoter sequence is operably linked to the nucleic acid sequence encoding the soluble sFasL polypeptide.

26. The method of claim 25, wherein the promoter is a ubiquitous promoter.

27. The method of claim 25, wherein the promoter is a tissue-specific promoter and/or a cell-specific promoter.

28. The method of claim 25, wherein the promoter promotes expression of a transgene in one or more cells within the retina (e.g., retinal pigment epithelial cells, retinal ganglion cells, photoreceptors, immune cells, etc.).

29. The method of any one of claims 1 to 28, wherein the nucleic acid molecule is a nucleic acid expression vector.

30. The method of claim 29, wherein the nucleic acid expression vector is a viral vector.

31. The method of claim 30, wherein the viral vector is selected from the group consisting of: an adenovirus vector, an adeno-associated virus vector, a lentivirus vector, a herpesvirus vector, a poxvirus vector, a baculovirus vector, a papillomavirus vector, and a papovavirus vector.

32. The method of any one of claims 28 to 31, wherein a viral particle or viral-like particle comprising the viral vector is administered to the eye.

33. The method of any one of claims 1 to 32, wherein the nucleic acid molecule is administered to the vitreous of the eye.

34. The method of any one of claims 1 to 32, wherein the nucleic acid molecule formulated within a composition suitable for administration to the vitreous of the eye.

35. A method of treating retinal pigment epithelial (RPE) cell death or a disorder associated therewith in an eye of an individual, the method comprising:
administering a sFasL therapeutic to the eye, wherein the sFasL therapeutic increases an amount of sFasL polypeptide in the eye.

36. A method of treating photoreceptor cell death in an eye of an individual having retinal pigment epithelial (RPE) cell death, the method comprising:
administering a sFasL therapeutic to the eye, wherein the sFasL therapeutic increases an amount of sFasL polypeptide in the eye.

37. The method of any one of claims 35 to 36, wherein the sFasL polypeptide comprises SEQ ID NO: 3.

38. The method of any one of claims 35 to 37, wherein the sFasL therapeutic comprises the sFasL polypeptide.

39. The method of any one of claims 35 to 37, wherein the sFasL therapeutic comprises a nucleic acid molecule encoding the sFasL polypeptide.

40. The method of any one of claims 35 to 37, wherein the sFasL therapeutic comprises a composition comprising a nucleic acid molecule encoding the sFasL polypeptide and carrier comprising the nucleic acid molecule encoding the sFasL polypeptide.


SEQUENCE LISTING (4 sequences)

SEQ ID NO: 1 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 2 - protein, 154 aa, Homo sapiens
EKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 3 - DNA, 465 bp, synthetic construct
  source 1..465: mol_type = other DNA
gagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 4 - protein, 735 aa, Adeno-associated dependoparvovirus A [sequence omitted]